(19) United States
(12) Patent Application Publication
(10) Pub. No.: US 2024/0066269 A1
(43) Pub. Date: Feb. 29, 2024

(54) Integrated Midline Catheter System

(71) Applicant: Becton, Dickinson and Company, Franklin Lakes, NJ (US)

(72) Inventor: Jonathan Karl Burkholz, Salt Lake City, UT (US)

(21) Appl. No.: 17/893,825

(22) Filed: Aug. 23, 2022

(51) Int. Cl. A61M 25/06

(52) U.S. Cl. CPC A61M 25/0606; A61M 25/06

(57) ABSTRACT

An integrated midline catheter system includes a catheter adapter having a catheter, a body receiving the catheter, and a side port in fluid communication with the catheter, a near patient access port in fluid communication with the side port of the catheter adapter, with the near patient access port including a connector portion configured to be coupled to a peripheral probe device, and a catheter insertion device including a housing, an introducer needle extending distally from the housing, and a guidewire advancement tab moveable relative to the housing to advance a guidewire through the introducer needle. The catheter adapter is configured to be advanced distally relative to the housing.

### BACKGROUND OF THE INVENTION

### Field of the Invention

The present disclosure relates to an integrated midline catheter system.

### Description of Related Art

Midline catheters are generally used for parenteral nutrition, IV fluid replacement, and/or the administration of analgesics and antibiotics. Midline catheters are inserted at the bedside using sterile techniques and can remain in place for several weeks. The insertion (venipuncture) is performed above and below the antecubital fossa in the cephalic, basilica, or bronchial veins. The catheter may be of various lengths and gauges, with the tip of the catheter terminating below the axilla and proximal central veins.

The potential advantages of a midline catheter are the reduced frequency of repeated venipunctures for labs/restarts, decreased incidence of catheter related infections, extended implant/indwell duration, improved clinical outcomes, patient satisfaction and associated cost savings. Placing the catheter tip in the larger diameter veins of the upper arm compared to smaller veins provides for improvements in drug delivery therapy and hemodilution. Midline catheters can also be used for infusing contrast media at higher flow rates that are typically done by other catheters such as, e.g., peripheral intravenous catheters (PIVCs).

However, an advantage of vascular access devices such as PIVCs over midline catheters is the recent development of systems to aid in blood draw and/or in-vein digital measurements directly from an indwelling catheter of the vascular access device. For example, a blood draw device known as PIVO™ from Becton, Dickinson and Company, is configured as a single-use device which temporarily attaches to a PIVC to draw a blood sample. Using an existing peripheral intravenous line as a conduit to the vasculature, the PIVO™ device advances a flexible, internal probe or flow tube through the PIVC to (or beyond) the catheter tip to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded. An example of one such blood draw device is shown and described in U.S. Pat. No. 10,300,247 B2, which is incorporated by reference herein in its entirety. Similarly, devices configured with direct probes, wires, fibers, guidewires, sensors, etc. through the PIVC and into the patient's vasculature for in-vein digital measurements have also been developed. On the other hand, conventional midline catheter systems lack the access architecture needed for compatibility with such blood draw and/or in-vein digital measurement devices.

### SUMMARY OF THE INVENTION

In one aspect or embodiment, an integrated midline catheter system includes a catheter adapter having a catheter, a body receiving the catheter, and a side port in fluid communication with the catheter, a near patient access port in fluid communication with the side port of the catheter adapter, with the near patient access port including a connector portion configured to be coupled to a peripheral probe device, and a catheter insertion device including a housing, an introducer needle extending distally from the housing, and a guidewire advancement tab moveable relative to the housing to advance a guidewire through the introducer needle. The catheter adapter is configured to be advanced distally relative to the housing.

The guidewire advancement tab may be positioned closer to a proximal end of the housing of the catheter insertion device than a distal end of the housing of the catheter insertion device. The catheter insertion device may include a handle. The near patient access port may further include a secondary port, with the secondary port being coupled to an integrated extension set. The system may further include a proximal access port coupled to a proximal end portion of the integrated extension set. The proximal access port may be color-coded to provide a flow rate indication. The proximal access port may include indicia indicative of at least one of catheter length and catheter gauge. The connector portion of the near patient access port may be a needle-free connector.

The catheter adapter may include a stabilization platform. The stabilization platform may be a pair of stabilizing wings.

The catheter insertion device may be detachable from the catheter adapter after insertion of the catheter within a patient's vasculature. The catheter may include a reinforced tip. The catheter adapter may include a strain relief.

The handle may be positioned closer to a distal end of the housing the catheter insertion device than a proximal end of the housing of the catheter insertion device. The handle may be configured to be grasped between a thumb and index finger of a clinician.

### BRIEF DESCRIPTION OF THE DRAWINGS

FIG. 1 is a perspective view of an integrated midline catheter system according to one aspect or embodiment of the present application;

FIG. 2 is a perspective view of an integrated midline catheter, stabilization, and near patient access port of FIG. 1;

FIG. 3 is a perspective view of an integrated midline catheter according to a further aspect or embodiment of the present application; and

FIG. 4 is a perspective view of an integrated midline catheter system according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

Embodiments of the present disclosure will primarily be described in the context of devices for use with integrated midline catheters. However, embodiments of the present disclosure equally extend to use with other catheter devices.

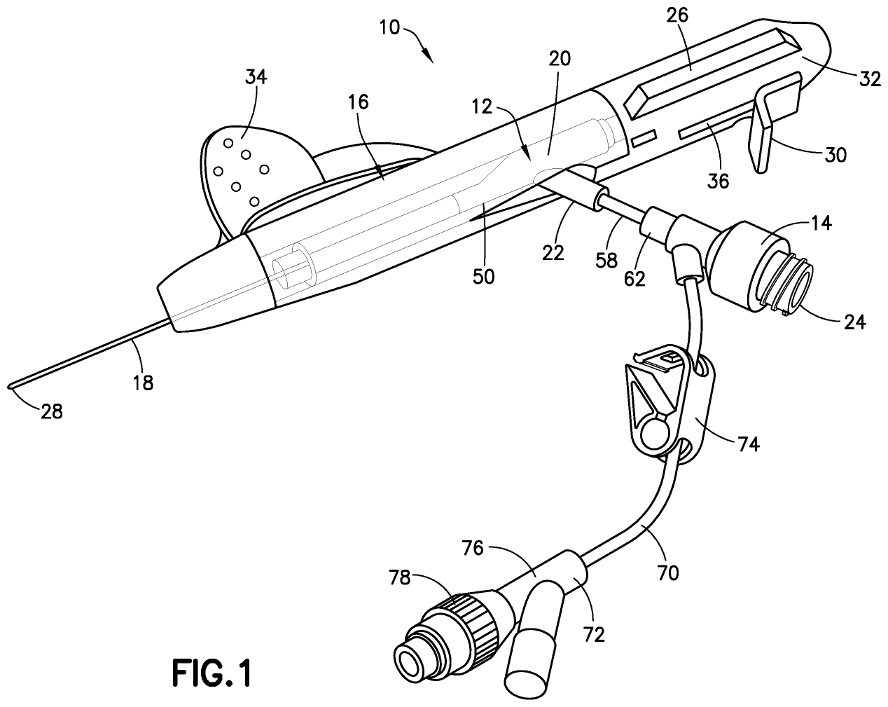

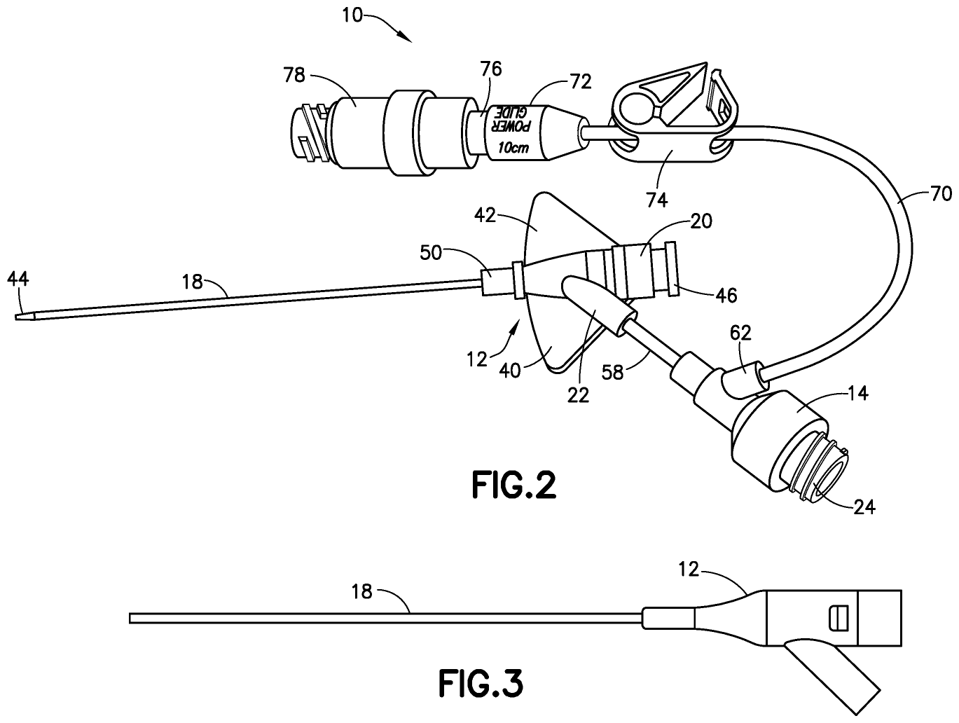

Referring to FIGS. 1 and 2, in one aspect or embodiment, an integrated midline catheter system 10 includes a catheter adapter 12, a near patient access port 14, and a catheter insertion device 16. The catheter adapter 12 includes a catheter 18, a body 20 receiving the catheter 18, and a side port 22 in fluid communication with the catheter 18. The near patient access port 14 is in fluid communication with the side port 22 of the catheter adapter 12. The near patient access port 14 includes a connector portion 24 configured to be coupled to a peripheral probe device. The catheter insertion device 16 includes a housing 26, an introducer needle 28 extending distally from the housing 26, and a guidewire advancement tab 30 moveable relative to the housing 26 to advance a guidewire through the introducer needle 28. The catheter adapter 12 is configured to be advanced distally relative to the housing 26. The integrated midline catheter system 10 is configured to provided full midline catheter features and integrated near patient access port architecture to provide a closed system midline that enables instrument, tubing, and probe delivery through the near patient access port 14 and into a patient's vascular system. The system 10 may be used for peripheral vascular access, IJ, EJ, and/or arterial access for blood collection or in-vein digital measurement.

Referring to FIG. 1, the guidewire advancement tab 30 is positioned at a proximal end 32 of the housing 26 of the catheter insertion device 16. The catheter insertion device 16 also includes a handle 34 for a clinician to stably hold the catheter insertion device 16 while operating the device 16. The handle 34 is configured to facilitate a clinician holding the handle 34 with one hand between a thumb and index finger while allowing the opposite hand to operate a guidewire and advance a catheter. The guidewire advancement tab 30 is positioned on a surface of the housing 26, with the guidewire advancement tab 30 configured for substantially linear movement along a slot 36 formed in the housing 26. While not shown in FIG. 1, the guidewire advancement tab 30 may be operably coupled to a guidewire, with the guidewire being sized and configured to selectively pass through (and beyond) the introducer needle 28 as the guidewire advancement tab 30 is advanced distally along the slot 36 and, conversely, to retract through the introducer needle 28 when the guidewire advancement tab 30 is retracted proximally by the clinician. The guidewire may include a blunt, atraumatic tip to substantially prevent vascular damage. In some embodiments, a polymer rod or tubing with an atraumatic tip may replace the guidewire.

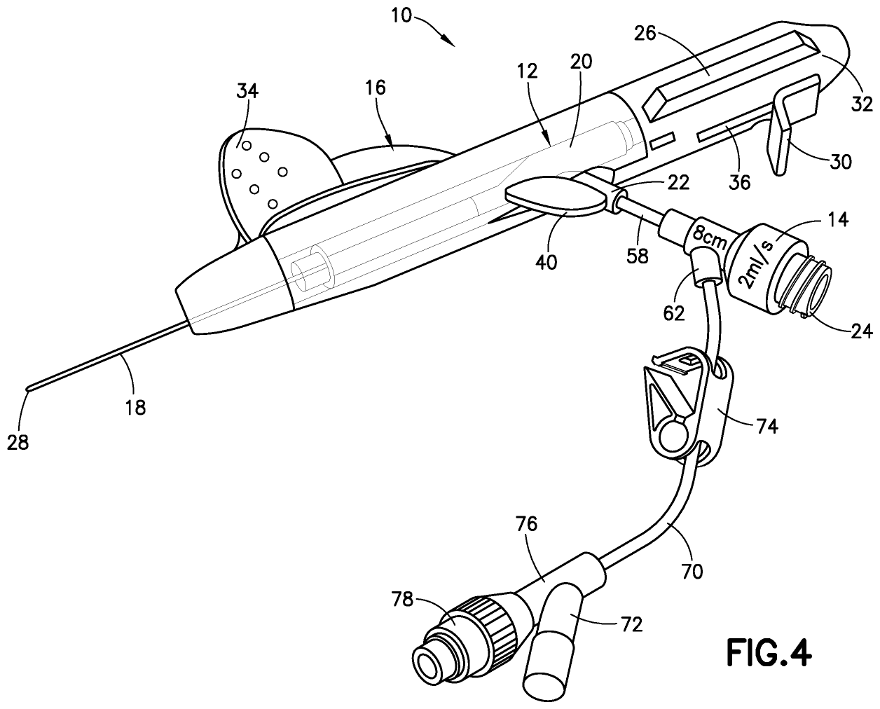

Referring to FIGS. 2 and 4, in some aspects or embodiments, the catheter adapter 12 or system 10 includes a stabilization platform formed by one or more stabilization wings and may include opposing stabilizing wings 40, 42. As shown in FIG. 4, the catheter adapter 12 is disposed at least partially within the housing 26 such that the stabilizing wings 40, 42 extend externally from opposing lateral sides of the housing 26. The catheter adapter 12 is configured to move linearly along the housing 26 in a distal direction such that the catheter 18 may be selectively advanced over the introducer needle 28 and into the patient's vasculature. The catheter 18 may be any appropriate length and gauge for use as a midline catheter. For example, catheter 18 may have a length between 8 cm and 10 cm, and may be one of 16 GA, 18 GA, 20 GA, or 22 GA. However, it is to be understood that catheter 18 is not limited to the above-referenced lengths and/or gauges. Additionally, the catheter 18 may be formed of any appropriate material such as, e.g., polyurethane. The catheter 18 may have a reinforced tip 44, which may be configured to substantially prevent the catheter tip from collapsing and/or aid in deployment and positioning of the catheter 18.

Referring to FIG. 3, in some aspects or embodiments, the catheter adapter 12 does not include a stabilization platform.

Placement of the catheter 18 into the patient's vasculature utilizing the system 10 will now be explained in accordance with an embodiment of the present disclosure. First, the clinician identifies the appropriate insertion site, and cleans and prepares the insertion site in accordance with institutional policy. The clinician then grips and advances the entire catheter insertion device 16 to insert the introducer needle 28 into an appropriate vein or other vascular access location of the patient. While not shown, the system 10 may include magnetic needle guidance to ensure proper insertion of the introducer needle 28. The magnetic needle guidance may be used in conjunction with ultrasound placement system and methods such as, e.g., the Cue™ Needle Tracking System from Becton, Dickinson and Co. With the introducer needle 28 in place, the clinician may then advance the guidewire advancement tab 30 distally along the slot 36 such that a guidewire (not shown) operably coupled to the guidewire advancement tab 30 simultaneously advances through the introducer needle 28, thereby providing an extended guided path for deployment of the catheter 18.

Next, with the guidewire in an advanced position, the clinician may distally advance the catheter adapter 12 along the housing 26, thereby also advancing the catheter 18 over the introducer needle 28 and guidewire, moving the catheter 18 into a desired position within the patient's vasculature. To distally advance the catheter adapter 12, the clinician may grip or otherwise manipulate one or both of the stabilizing wings 40, 42. Additionally and/or alternatively, the clinician may utilize the side port 22 extending from the catheter adapter 12 to aid in advancement of the catheter adapter 12 and the catheter 18.

With the catheter 18 and the catheter adapter 12 in a desired position, the clinician may separate the catheter insertion device 16 from the catheter adapter 12. In doing so, the introducer needle 28 and guidewire (not shown) are also withdrawn from the catheter 18 and are pulled through a self-sealing proximal connector portion 46 of the catheter adapter 12. Accordingly, with the catheter insertion device 16 removed, the catheter adapter 12 remains in place at the insertion site in the configuration shown in FIG. 2, with the stabilizing wings 40, 42 configured to stabilize the catheter adapter 12. In some embodiments, a strain relief feature 50 may be provided between the catheter adapter 12 and the catheter 18. In one aspect or embodiment, the catheter insertion device 16 is the same or similar to the catheter insertion device 16 shown and described in U.S. Patent Application Publication No. 20200001051 to Huang et al., which is hereby incorporated by reference in its entirety.

Referring again to FIGS. 1 and 2, the near patient access port 14 is configured to provide catheter access to peripheral devices such as, e.g., a blood draw device (e.g., PIVO™ from Becton, Dickinson and Company), or a vascular access probe (VAP) for in-vein digital measurement of patient date such as temperature, pH, lactate, and/or other blood-based measurements. In some embodiments, the near patient access port 14 is coupled to the side port 22 of the catheter adapter 12 via a length of intermediate tubing 58. However, in other embodiments, the near patient access port 14 may be coupled directly to the side port 22, or connected via another intermediate member.

The near patient access port 14 includes the connector portion 24, which in some embodiments, is configured to be compatible with peripheral devices such as blood draw devices and/or vascular access probes. The connector portion 24 may include an interface for secure coupling of the peripheral device(s) to the connector portion 24. In some embodiments, the connector portion 24 is configured as a needle-free connector (NFC) configured to receive, e.g., a blunt introducer of a blood draw device. More specifically, the connector portion 24 may be configured as a split-septum NFC with direct probe access such as, e.g., Q-Syte™ or SmartSite™ NFCs from Becton, Dickinson and Co., or any other appropriate split-septum NFC. Alternatively, in other embodiments, the connector portion 24 may be formed of a non-split-septum-type NFC. Furthermore, in some embodiments, the near patient access port 14 may include anti-microbial and/or flush-promoting features. For example, the near patient access port 14 may include one or more of an offset tubing port vortex-creating feature, a proximal flow-diverting feature, anti-microbial NFC lubricant, anti-microbial eluting surface coating(s) or insert(s), etc.

With the near patient access port 14 fluidly coupled to the catheter adapter 12 via the side port 22, the system 10 provides for probe (or tube) access from a peripheral probe device through the indwelling catheter 18. As catheter 18 is a midline catheter and is generally longer in length, e.g., a PIVC, the length of the probe or tube of the peripheral probe device may be altered and/or optimized for use with the midline catheter, thereby enabling the probe or tube to potentially extend beyond the reinforced tip 44 of the catheter 18 when deployed.

The near patient access port 14 further includes a secondary port 62 positioned near a distal end thereof. In some embodiments, the secondary port 62 is coupled to an integrated extension set 70, with the integrated extension set 70 further being coupled to a proximal portion 72 at a proximal end thereof. A clamp 74 may be provided on the integrated extension set 70, with the clamp 74 configured to selectively restrict flow through the integrated extension set 70. In some embodiments, the clamp 74 may be color-coded to signify the type and/or injection compatibility of the integrated extension set 70.

In some embodiments, the proximal portion 72 may include a proximal access port 76 and a proximal connector 78. The proximal connector 78 may be removably or non-removably coupled to the proximal access port 76, and the proximal connector 78 may be configured to allow fluid infusion through the catheter 18 via the near patient access port 14. In some embodiments, the proximal access port 76 may be color-coded and/or may contain indicia to indicate catheter length, catheter gauge, high-pressure injection compatibility, etc. In other embodiments, during insertion of the catheter 18 into the patient's vasculature, the proximal connector 78 may be replaced with, e.g., a removable vent plug. In some embodiments, the proximal access port 76 may be a non-split-septum connector, and may include antimicrobial and/or flush-ability features. While only a single proximal access port 76 is shown, it is to be understood that the proximal portion 72 may be configured to include more than one access port.

In the embodiments shown in FIGS. 1-4, the secondary port 62 of the near patient access port 14 is configured as an angled port, resulting in the near patient access port 14 being a y-shaped adapter. However, in other embodiments, the secondary port 62 may be configured as a t-shaped adapter such that the secondary port 62 enters the near patient access port 14 at a substantially 90° angle. Additionally, while secondary port 62 is shown as being directed towards the center of the device, it is to be understood that secondary port 62 may be directed away from the device.

Accordingly, the integrated midline catheter system 10 described above provides a midline catheter system with a near patient access port compatible with an instrument, probe, and/or tubing delivery through the midline catheter and into the patient's vascular system, which conventional integrated midline catheter systems do not provide.

While several embodiments of integrated midline catheter systems having near patient access ports for peripheral probe device access were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. An integrated midline catheter system comprising:
a catheter adapter comprising a catheter, a body receiving the catheter, and a side port in fluid communication with the catheter;
a near patient access port in fluid communication with the side port of the catheter adapter, the near patient access port comprising a connector portion configured to be coupled to a peripheral probe device; and
a catheter insertion device comprising a housing, an introducer needle extending distally from the housing, and a guidewire advancement tab moveable relative to the housing to advance a guidewire through the introducer needle, wherein the catheter adapter is configured to be advanced distally relative to the housing.

2. The system of claim 1, wherein the guidewire advancement tab is positioned closer to a proximal end of the housing of the catheter insertion device than a distal end of the housing of the catheter insertion device.

3. The system of claim 1, wherein the catheter insertion device comprises a handle.

4. The system of claim 1, wherein the near patient access port further comprises a secondary port, and wherein the secondary port is coupled to an integrated extension set.

5. The system of claim 4, further comprising a proximal access port coupled to a proximal end portion of the integrated extension set.

6. The system of claim 5, wherein the proximal access port is color-coded to provide a flow rate indication.

7. The system of claim 5, wherein the proximal access port comprises indicia indicative of at least one of catheter length and catheter gauge.

8. The system of claim 1, wherein the connector portion of the near patient access port is a needle-free connector.

9. The system of claim 1, wherein the catheter adapter comprises a stabilization platform.

10. The system of claim 9, wherein the stabilization platform comprises a pair of stabilizing wings.

11. The system of claim 1, wherein the catheter insertion device is detachable from the catheter adapter after insertion of the catheter within a patient's vasculature.

12. The system of claim 1, wherein the catheter comprises a reinforced tip.

13. The system of claim 1, wherein the catheter adapter comprises a strain relief.

14. The system of claim 3, wherein the handle is positioned closer to a distal end of the housing the catheter insertion device than a proximal end of the housing of the catheter insertion device.

15. The system of claim 14, wherein the handle is configured to be grasped between a thumb and index finger of a clinician.
